(19) United States
(12) Patent Application Publication
(10) Pub. No.: US 2026/0232190 A1
(43) Pub. Date: Aug. 13, 2026

(54) PATIENT MONITORING DEVICE

(71) Applicant: Murata Vios, Inc., Woodbury, MN (US)

(72) Inventors: Nobuhiro KONDO, Woodbury, MN (US); Masamichi YANAI, Woodbury, MN (US); Atsuhiko HIRATA, Woodbury, MN (US); Masaaki KANAO, Nagaokakyo-shi (JP)

(21) Appl. No.: 19/531,970

(22) Filed: Feb. 6, 2026

Related U.S. Application Data
(60) Provisional application No. 63/757,003, filed on Feb. 11, 2025.

(51) Int. Cl. A61B 5/00; H01Q 1/27; H01Q 1/42

(52) U.S. Cl. CPC A61B 5/0002; H01Q 1/273; H01Q 1/42; A61B 2560/0214

(57) ABSTRACT

A patient-monitoring device includes a housing, an internal power source within the housing, and a replaceable battery pack that is pluggable with and unpluggable from the housing. The patient-monitoring device is configured to monitor a patient and to transmit vital sign data of the patient when the patient-monitoring device is attached to a body of the patient by being powered by the replaceable battery pack when the replaceable battery pack is plugged into the patient-monitoring device and by being powered by the internal power source when the replaceable battery pack is unplugged from the patient-monitoring device.

### CROSS REFERENCE TO RELATED APPLICATIONS

This application claims the benefit of U.S. Patent Application No. 63/757,003 filed on Feb. 11, 2025. The entire contents of this application are hereby incorporated by reference.

### BACKGROUND OF THE INVENTION

### 1. Field of the Invention

The present invention relates to patient monitoring devices. More specifically, the present invention relates to patient monitoring devices that are attached to the patient's body and that wirelessly send patient vital sign data by radio.

### 2. Description of the Related Art

It is known to use a patient monitoring devices that is attached to a patient's body and that is powered by disposable or rechargeable battery that requires periodic replacement. A problem with these known patient monitoring devices is that vital signs cannot be monitored during replacement of the disposable or rechargeable battery.

In addition, known patient monitoring devices that wirelessly transmit patient vital sign data have a risk of interfering with other medical devices, especially medical devices embedded in the patient, such as pacer makers.

### SUMMARY OF THE INVENTION

To overcome the problems described above, example embodiments of the present invention provide patient monitoring devices that include an internal power source, such as a small battery or capacitor, so that the patient monitoring devices can continuously monitor a patient's vital signs during battery exchanges.

According to an example embodiment of the present invention, a patient-monitoring device includes a housing, an internal power source within the housing, and a replaceable battery pack that is pluggable with and unpluggable from the housing. The patient-monitoring device is configured to monitor a patient and to transmit vital sign data of the patient when the patient-monitoring device is attached to a body of the patient by being powered by the replaceable battery pack when the replaceable battery pack is plugged into the patient-monitoring device and by being powered by the internal power source when the replaceable battery pack is unplugged from the patient-monitoring device.

The internal power source can have an electric capacity that can provide power to the patient-monitoring device so that the patient-monitoring device can monitor and transmit the vital sign data of the patient for at least 3 seconds after the replaceable battery pack is unplugged from the patient-monitoring device.

The internal power source can include a battery or a capacitor. The internal power source can include a rechargeable battery, a lithium-ion capacitor, an electrical double-layer capacitor, or a lithium-ion battery.

The patient-monitoring device can further include a DC/DC converter connected to the internal power source. The patient-monitoring device can further include an antenna within the housing, where the vital sign data of the patient can be transmitted via the antenna. When the patient-monitoring device is attached to the body of the patient, the internal power source can be located between the antenna and the body of the patient. An association between the patient and the patient-monitoring device can be maintained during a time period between when the replaceable battery pack is unplugged from the patient-monitoring device and when a new replaceable battery pack is plugged into the patient-monitoring device.

According to an example embodiment of the present invention, a patient-monitoring system includes the patient-monitoring device of one of the various other example embodiments of the present invention and includes a display that is separate from the patient-monitoring device and that displays the vital sign data of the patient.

The above and other features, elements, characteristics, steps, and advantages of the present invention will become more apparent from the following detailed description of example embodiments of the present invention with reference to the attached drawings.

### BRIEF DESCRIPTION OF THE DRAWINGS

FIG. 1 shows a patient monitoring device that is attached to a patient and that includes an antenna for sending patient vital sign data and a replaceable battery.

FIG. 2 shows an electrical block diagram of the patient monitoring device of FIG. 1 that includes a DC/DC converter and an internal power source.

FIG. 3 shows a block diagram of the mechanical structure of the patient monitoring device of FIG. 1 that shows the arrangement of the internal power source and the antenna.

### DETAILED DESCRIPTION OF EXAMPLE EMBODIMENTS

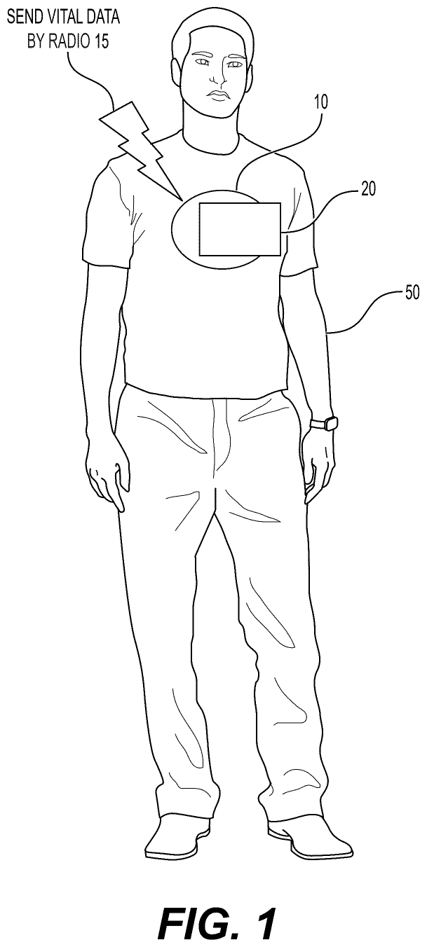

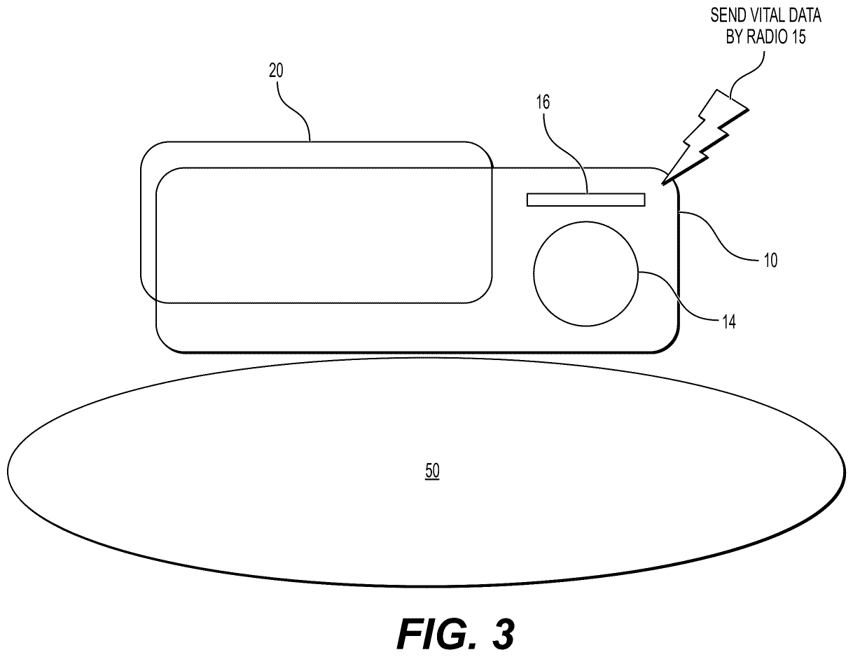

FIGS. 1 and 3 show a patient-monitoring device 10 attached to the body of a patient 50 so that the patient-monitoring device 10 can monitor the vital signs of the patient 50. For example, the patient-monitoring device 10 can be attached to an electrode attached to the patient 50 that receives electrical signals generated by the patient 50. The patient-monitoring device 10 can include a replaceable battery 20 that can be plugged into and unplugged from the patient-monitoring device 10. For example, the replaceable battery 20 might last 24 hours and then will need to be replaced once a day.

The patient-monitoring device 10 can be included in a patient-monitoring system that monitors vital signs of the patient 50. The patient-monitoring device 10 can monitor the vital signs of the patient 50 and can wirelessly transmit the patient vital sign data to a display of a patient-monitoring system that displays the vital signs of the patient 50 to, for example, a caregiver. For example, if the patient 50 is in a hospital, the patient-monitoring device 10 can transmit the patient vital sign data to a bedside monitor or to a nurse's station located within the hospital so that a nurse or other caregiver can monitor the patient 50, or if the patient 50 is home, then the patient-monitoring device 10 can transmit the patient vital sign data to a central location outside of the home so that a remote caregiver can monitor the patient 50. The patient-monitoring device 10 can also receive signals from other components of the patient-monitoring system.

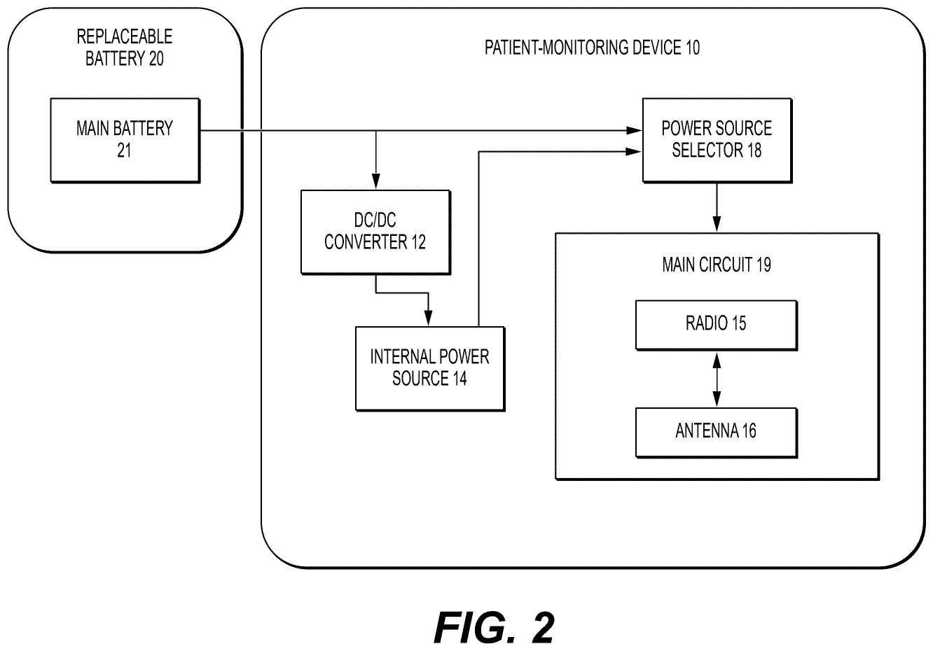

As shown in FIG. 2, the patient-monitoring device 10 can include an internal power source 14 that allows the patient-monitoring device 10 to continuously monitor the patient 50 during battery exchange. The internal power source 14 can be or can include a battery or can be capacitor. For example, the internal power source 14 can be or can include a rechargeable battery, or the internal power source 14 can be or can include a lithium-ion capacitor, a lithium-ion battery, electrical double-layer capacitor, or other suitable power source.

The patient-monitoring device 10 is configured to monitor a patient 50 and to transmit vital sign data of the patient 50 when the patient-monitoring device 10 is attached to a body of the patient 50 by being powered by the replaceable battery pack 20 when the replaceable battery pack 20 is plugged into the patient-monitoring device 10 and by being powered by the internal power source 14 when the replaceable battery pack 20 is unplugged from the patient-monitoring device 10. The internal power source 14 does not have to provide power to the patient-monitoring device 10 when the replaceable battery pack 20 is plugged into the patient-monitoring device 10. The internal power source 14 can have an electric capacity such that the internal power source 14 can provide power to the patient-monitoring device 10 so that the patient-monitoring device 10 monitors and transmits patient vital sign data of the patient 50 for at least 3 seconds after the replaceable battery pack 20 is unplugged from the patient-monitoring device 10.

As shown in FIG. 3, the internal power source 14 can be located between the antenna 16 and the body of the patient 50. This arrangement of the internal power source 14 being located between the antenna 16 and the body of the patient 50 allows the internal power source 14 to keep the antenna 16 sufficiently away from the patient 50, helping to avoid any undesirable impact of radio waves on the body of the patient 50.

The patient-monitoring device 10 includes a main circuit 19 that includes the circuitry used to monitor the vital signs of the patient 50. The main circuit 19 includes a radio 15 and an antenna 16 that can be used to transmit patient vital sign data. For example, the patient vital sign data can be transmitted using any suitable protocol, including, for example, WiFi or Bluetooth. The patient-monitoring device 10 can include DC/DC converter 12 that is connected to an internal power source 14 and can include a power source selector 18 that is connected to a main battery 21 in the replaceable battery 20 and that is connected to the internal power source 14. When the replaceable battery 20 is plugged into the patient-monitoring device 10, the power source selector 18 can select the main battery 21 of the replaceable battery 20 to power the main circuit 19. And when the replaceable battery 20 is unplugged from the patient-monitoring device 10, the power source selector 18 can select the internal power source 14 to power the main circuit 19.

When the replaceable battery 20 is plugged into the patient-monitoring device 10 (i.e., when the internal power source 14 is not providing power to the patient monitoring device 10), the main battery 21 can be used to charge the internal power source 14 through the DC/DC converter 12. If the voltage of the main battery 21 is too high or too low, then the DC/DC converter 12 can generate appropriate charging voltage for the internal power source 14. Specifically, if the voltage of the main battery 21 is too high, then the DC/DC converter 12 can step down the voltage of the main battery 21 to the appropriate charging voltage for the internal power source 14, and if the voltage of the main battery 21 is too low, then the DC/DC converter 12 can step up the voltage of the main battery 21 to the appropriate charging voltage for the internal power source 14. If the voltage of the main battery 21 provides the appropriate charging voltage for the internal power source 14, then the DC/DC converter 12 can pass-through the voltage from the main battery 21 to charge the internal power source 14. Any suitable DC/DC converter can be used for the DC/DC converter 12, including, for example, a buck-boost DC/DC converter. A benefit of using a buck-boost topology is that the internal power source 14 can be charged with enough voltage even when the main battery 21 is discharged.

The patient-monitoring device 10 can be associated with the patient 50. For example, a serial number of the patient-monitoring device 10 and a patient ID can be linked (i.e., patient association). The patient-monitoring system can include a remote computer or server that includes a database in memory that stores the serial number or other identifier of each patient-monitoring device 10 and a patient ID of each patient. When the patient-monitoring device 10 is attached to a patient, the patient-monitoring device 10 can be associated with the patient 50 by linking the serial number or other identifier of the patient-monitoring device 10 and the patient ID of the patient 50. This association can be done by the patient or by the caregiver. The linking of the serial number or other identifier of the patient-monitoring device 10 and the patient ID of the patient 50 results in the serial number or other identifier and the patient ID being linked in the database.

The patient-monitoring device 10 and the patient 50 can be disassociated by de-linking the serial number or other identifier of the patient-monitoring device 10 and the patient ID of the patient 50 in the database. This disassociating significantly reduces or minimizes the possibility, when the patient-monitoring device 10 is attached to a new patient, of mixing up the patients and displaying the vital signs of one patient for another patient.

If the patient-monitoring device 10 stops transmitting patient vital sign data, then the patient 50 and the patient-monitoring device 10 can be disassociated. The disassociating can be immediate or after some fixed time period (e.g., 10 minutes or other suitable time period). For example, if the patient-monitoring device 10 stops transmitting the patient vital sign data because the patient-monitoring device 10 has been removed from the patient 50, then the patient-monitoring system can disassociate the patient 50 and the patient-monitoring device 10, which can significantly reduce or minimize the possibility, when the patient-monitoring device 10 is attached to a new patient, of mixing up the patients because the patient association remains unchanged.

The replaceable battery 20 must be periodically replaced, and without the internal power source 14, the patient-monitoring device 10 can stop transmitting patient vital sign data, resulting in the patient 50 and the patient-monitoring device 10 being disassociated every time the replaceable battery 20 is replaced. The patient-monitoring system might also disassociate the patient 50 while the patient-monitoring device 10 is transmitting data, if patient-monitoring system detects that the ECG leads of the patient-monitoring device 10 are no longer connected to the patient 50. This dissociation when detecting the ECG leads are no longer connected can significantly reduce or minimize the risk of a patient-association mix up. Alternatively, the patient-monitoring device 10 might be programmed to turn off when a fixed time period passes after the ECG leads are off. Then, the patient-monitoring system will disassociate the patient from the patient-monitoring device 10 after the fixed time period.

These disassociations require reassociations, which can be time-consuming and inconvenient. Therefore, if the internal power source 14 can power the patient-monitoring device 10 long enough for the replaceable battery 20 to be replaced, then the caregiver's job can be made easier because reassociation does not have to be performed and significantly reduce or minimize the risk of mixing up the patients and displaying the vital signs of one patient for another patient.

To re-associate to a new patient, the patient-monitoring device 10 can be turned off to trigger disassociation or the ECG terminals can be removed from the patient 50 to cause disassociation, either immediately or a fixed time period (e.g., 30 seconds or other suitable fixed time period). The dissociation allows a new association to be performed with a new patient. After being used with a patient, the patient-monitoring device 10 must be cleaned, which lasts longer than backup battery, before being attached to a new patient.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1. A patient-monitoring device comprising:
a housing;
an internal power source within the housing; and
a replaceable battery pack that is pluggable with and unpluggable from the housing; wherein
the patient-monitoring device is configured to monitor a patient and to transmit vital sign data of the patient when the patient-monitoring device is attached to a body of the patient by being powered by:
the replaceable battery pack when the replaceable battery pack is plugged into the patient-monitoring device; and
the internal power source when the replaceable battery pack is unplugged from the patient-monitoring device.

2. The patient-monitoring device of claim 1, wherein the internal power source has an electric capacity that can provide power to the patient-monitoring device so that the patient-monitoring device monitors and transmits the vital sign data of the patient for at least 3 seconds after the replaceable battery pack is unplugged from the patient-monitoring device.

3. The patient-monitoring device of claim 1, wherein the internal power source includes a battery or a capacitor.

4. The patient-monitoring device of claim 1, wherein the internal power source includes a rechargeable battery, a lithium-ion capacitor, an electrical double-layer capacitor, or a lithium-ion battery.

5. The patient-monitoring device of claim 1, further comprising a DC/DC converter connected to the internal power source.

6. The patient-monitoring device of claim 1, further comprising an antenna within the housing; wherein
the vital sign data of the patient is transmitted via the antenna.

7. The patient-monitoring device of claim 6, wherein, when the patient-monitoring device is attached to the body of the patient, the internal power source is located between the antenna and the body of the patient.

8. The patient-monitoring device of claim 1, wherein an association between the patient and the patient-monitoring device is maintained during a time period between when the replaceable battery pack is unplugged from the patient-monitoring device and when a new replaceable battery pack is plugged into the patient-monitoring device.

9. A patient-monitoring system comprising:
the patient-monitoring device of claim 1; and
a display that is separate from the patient-monitoring device and that displays the vital sign data of the patient.

10. The patient-monitoring system of claim 9, wherein the internal power source has an electric capacity that can provide power to the patient-monitoring device so that the patient-monitoring device monitors and transmits the vital sign data of the patient for at least 3 seconds after the replaceable battery pack is unplugged from the patient-monitoring device.

11. The patient-monitoring system of claim 9, wherein the internal power source includes a battery or a capacitor.

12. The patient-monitoring system of claim 9, wherein the internal power source includes a rechargeable battery, a lithium-ion capacitor, an electrical double-layer capacitor, or a lithium-ion battery.

13. The patient-monitoring system of claim 9, further comprising a DC/DC converter connected to the internal power source.

14. The patient-monitoring system of claim 9, further comprising an antenna within the housing; wherein
the vital sign data of the patient is transmitted via the antenna.

15. The patient-monitoring system of claim 14, wherein, when the patient-monitoring device is attached to the body of the patient, the internal power source is located between the antenna and the body of the patient.

16. The patient-monitoring system of claim 9, wherein an association between the patient and the patient-monitoring device is maintained during a time period between when the replaceable battery pack is unplugged from the patient-monitoring device and when a new replaceable battery pack is plugged into the patient-monitoring device.
